(19) United States
(12) Patent Application Publication
(10) Pub. No.: US 2022/0275420 A1
(43) Pub. Date: Sep. 1, 2022

(54) ENZYMATIC BIOCATHODE, METHOD FOR PRODUCING IT AND FUEL BIOCELL AND BIOSENSOR COMPRISING THIS ENZYMATIC BIOCATHODE

(71) Applicants: UNIVERSITÉ GRENOBLE ALPES, Saint Martin d'Heres (FR); INSTITUT NATIONAL DE LA SANTÉ ET DE LA RECHERCHE MEDICALE - INSERM, Paris (FR); GRENOBLE INP (INSTITUT NATIONAL POLYTECHNIQUE), Grenoble (FR); CNRS - CENTRE NATIONAL DE LA RECHERCHE SCIENTIFIQUE, Paris (FR)

(72) Inventors: Abdelkader ZEBDA, Grenoble (FR); Philippe CINQUIN, Saint Nazaire Les Eymes (FR); Donald MARTIN, Gieres (FR); Thomas SORANZO, Grenoble (FR)

(73) Assignees: UNIVERSITÉ GRENOBLE ALPES, Saint Martin d'Heres (FR); INSTITUT NATIONAL DE LA SANTÉ ET DE LA RECHERCHE MEDICALE - INSERM, Paris (FR); GRENOBLE INP (INSTITUT NATIONAL POLYTECHNIQUE), Grenoble (FR); CNRS - CENTRE NATIONAL DE LA RECHERCHE SCIENTIFIQUE, Paris (FR)

(21) Appl. No.: 17/632,094

(22) PCT Filed: Jul. 27, 2020
(86) PCT No.: PCT/IB2020/057059

§ 371 Date: Feb. 1, 2022

(30) Foreign Application Priority Data
Aug. 1, 2019 (FR) 1908856

(51) Int. Cl. C12Q 1/00; C12Q 1/26; H01M 4/86; H01M 4/90; H01M 8/16

(52) U.S. Cl. CPC C12Q 1/003; C12Q 1/26; H01M 4/8605; H01M 4/9008; H01M 8/16

(57) ABSTRACT

A biomass-based enzymatic biocathode based on glucose, monosaccharide, ketone or aldehyde includes a collector conductor support, conductive particles disposed on and bound to said collector conductor support, and an aldose reductase disposed on said conductive particles, being bound thereto by adsorption and accessible at the surface of the monosaccharide, ketone or aldehyde reagent that is to be reduced when the biocathode is operational.

The present invention relates to an enzymatic biocathode, a method for producing it, as well as a fuel biocell comprising it for its application to energy conversion and a biosensor comprising it for its application to sensing.

A fuel cell is an electrical cell which, unlike storage cells, can be supplied with a fuel continuously so that the electrical power output is sustained indefinitely (CONNIHAN M. A., (1981) Dictionary of Energy, Routledge and Kegan Paul). They convert chemical energy from fuel into electrical energy by the electrochemical reactions of fuel and an oxidant. A fuel cell consists of two electrodes—an electron-emitting anode and an electron-receiving cathode-separated by an electrolyte that allows the passage of ions. At the anode and cathode respectively, oxidation of the fuel (anode) and reduction of the oxidant (cathode) take place. The oxidation and reduction reactions at the electrodes require the use of metallic or molecular catalysts.

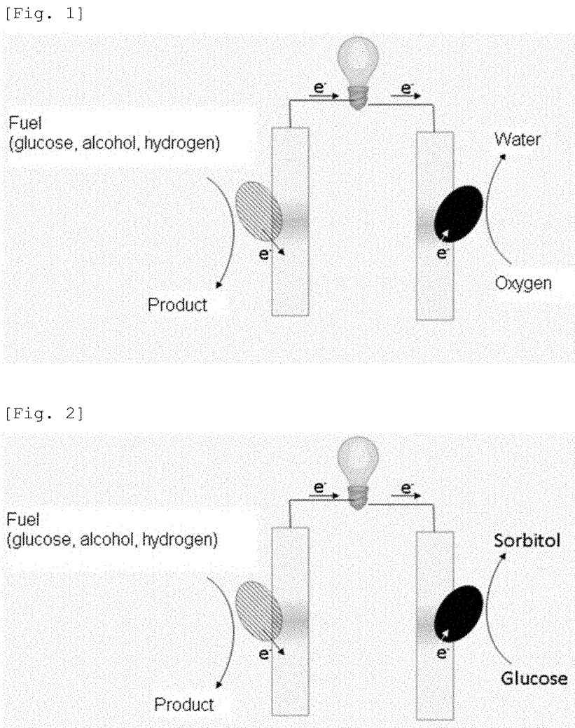

An Enzymatic Biofuel Cell (EBFC) is a sub-class of fuel cells, relying on purified redox enzymes to perform electrocatalytic reactions (see FIG. 1 in the attached drawing). When considering power output, EBFCs cannot compete with conventional fuel cells, which are capable of delivering power densities up to 1 W/cm², 1000 times higher than those delivered by enzymatic bio-cells. However, compared to metal catalysts, the attractiveness of enzymes lies in their high specificity towards their respective substrates and their ability to achieve high catalytic yields under mild conditions (20-40° C. in a reasonable pH range of 5-8 or even at neutral pH). Therefore, such electrochemical generators are envisaged to operate in complex media such as physiological fluids or plants.

In the case of enzymatic fuel cells, the most widely studied fuel is glucose, but other important fuels such as ethanol, lactate or glycerol are also new fuels for collecting energy from biomass.

The oxidant is often dioxygen or hydrogen peroxide, as both these substrates have a higher reduction potential. Indeed, the voltage of a fuel cell is the difference between the reduction potential of the oxidant and the oxidation potential of the fuel. Thus, to function, a fuel cell must use an oxidant with a higher reduction potential than the oxidation potential of the fuel. For this reason, the number of oxidants used is very limited, as few substrates have a high reduction potential. Furthermore, in the case of implantable biofuel, the only oxidant available in a living body is oxygen. Moreover, the available dioxygen is limited by its low concentration due to its low solubility in water: 0.2 mM and only 0.05 mM in a living body (Challenges for successful implantation of biofuel cells, A. Zebda, J-P Alcaraz, P. Vadgama, S. Shleev, P. Cinquin, D. K. Martin, Bioelectrochemistry, pages 57-72 (2018).

The low dioxygen concentration often leads to the biocathode being the limiting electrode in bio-cells.

Implantable and even non-implantable biofuel cells are therefore limited in performance by the low dioxygen concentration in a physiological fluid (0.05 mM) or water (0.2 mM). Indeed, the dioxygen concentration is 100 times lower than the glucose level in the body (5 mM), which presents a major problem as this low dioxygen concentration limits the performance in terms of electrical power output. Shleev et al (S. Shleev, Quo Vadis, Implanted Fuel Cell ChemPlusChem 2017, 82, 522-539) demonstrated that, in the case of implantable glucose biofuel cells, the low dioxygen concentration limits the current density delivered by the glucose biofuel cell to 40 μA/cm². Thus, increasing the performance of the implantable glucose biofuel cell requires the use of a cathode with a high surface area. For example, to power a medical device consuming 1 mW, a glucose biofuel cell operating at 300 mV must have a biocathode with a surface area of about 400 cm², which is, from a medical point of view, a serious constraint to the space available in the body for the implantation of the glucose biofuel cell. For this reason, it is known that in the case of implantable glucose biofuel, the low oxygen concentration associated with its low water solubility (0.2 mM) causes the biocathode to be the limiting electrode in the biofuel cell.

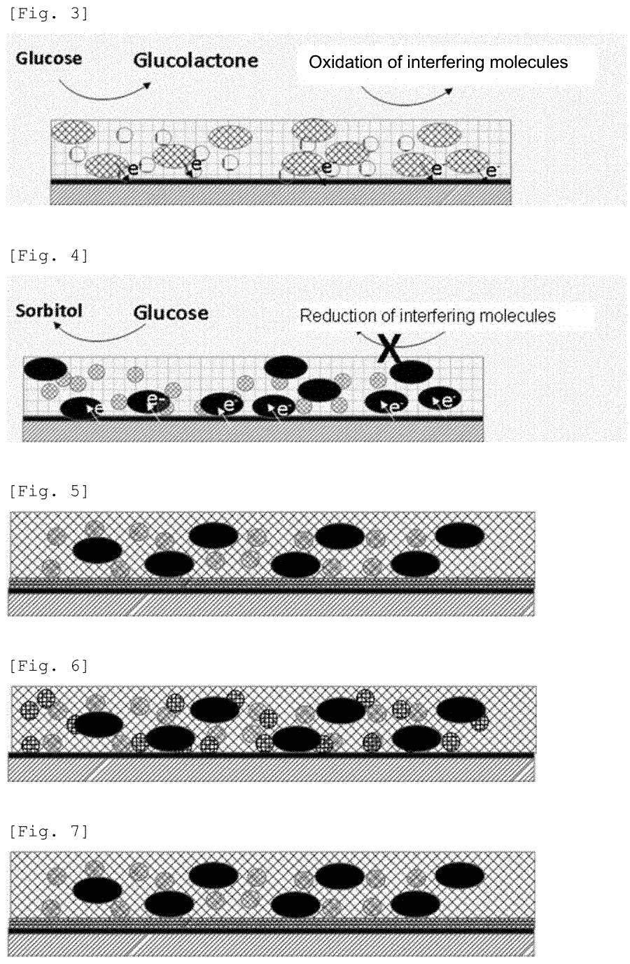

Existing glucose biosensors are based on the measurement of glucose oxidation current often at a positive potential. These glucose biosensors suffer from the oxidation current of interfering molecules present in a physiological fluid, which decreases the sensitivity of the biosensors (see FIG. 3 in the attached drawing).

The present invention aims to overcome the above disadvantages of bio-cells and biosensors and to this end proposes a new enzymatic biocathode architecture capable of using glucose or aldehyde as an oxidant (see FIG. 2) with glucose reduction catalysis at a relatively high voltage.

Thus, the enzymatic biocathode according to the invention is capable of converting the chemical energy of glucose or an aldehyde into electrical energy via an enzymatic reduction of glucose or the aldehyde. Indeed, this biocathode uses glucose or an aldehyde as an oxidant, allowing the construction of an implantable glucose biofuel cell that operates at 100% on glucose without the need for dioxygen.

The use of glucose as an oxidant in an implantable glucose biofuel enables the performance of the biofuel cell to be increased considerably. In addition, the implantable glucose biofuel cell can be used as an energy source to produce dioxygen in-vivo.

The biocathode according to the invention can also be used to design a biofuel cell operating under anaerobic conditions (e.g. underwater, mines, special conditions). In this case, the fuel cell will use glucose or other biomass compound as oxidant at the biocathode and also as reductant at the bioanode.

Furthermore, the biocathode according to the invention offers the possibility to measure a glucose level at a very low potential by measuring a glucose reduction current, which prevents interference from oxidation of interfering molecules. The biocathode operates in a reduction mode at a potential far from the oxidation potential of interfering molecules present in a physiological fluid, thereby increasing the sensitivity and lifetime of the biosensor (see FIG. 4).

The present invention is therefore primarily concerned with a biomass-based enzymatic biocathode based on monosaccharide, ketone or aldehyde characterised by the fact that it comprises:
a collector conductor support;
conductive particles disposed on and bound to said collector conductor support;
an aldose reductase disposed on said conductive particles, being bound thereto by adsorption and being accessible at the surface for the monosaccharide, ketone or aldehyde reagent to be reduced when the biocathode is operational.

The collector conductor support can advantageously be selected from:
continuous sheets of carbon, graphene or graphite;
continuous sheets of a metal, such as gold;
continuous indium tin oxide (ITO) sheets; and
carbon fibre non-woven fabrics.

The conductive particles can advantageously be selected from carbon, graphene, graphite, carbon black or mesoporous carbon nanotubes, in particular of multiwalled carbon nanotubes (MWCNT).

To the aldose reductase is advantageously associated its nicotinamide adenine dinucleotide phosphate (NADPH) cofactor, in which case the biocathode may comprise at least one agent for the regeneration of said NADPH cofactor by catalysing its reduction at the surface of the biocathode, said regeneration agent allowing an electro- or a photo-regeneration, being in this case photosensitive.

The regeneration agent may be an agent for the electroregeneration of the NADPH cofactor at the surface of the biocathode, said electroregeneration agent being at least one redox polymer selected in particular from benzylpropylviologen, a viologen polysiloxane polymer, polyaniline or polypyrrole.

The regeneration agent may be a photosensitive agent for the regeneration of the NADPH cofactor at the surface of the biocathode, said photosensitive agent being at least one redox photosensitive polymer chosen in particular from methylene green, methylene blue, neutral red, polyaniline and polypyrrole polymers.

The photosensitive agent may also be at least one non-polymeric photosensitive compound selected in particular from chlorophyll, acridine, (pentamethylcyclopentadienyl-2,2V-bipyridine aqua) rhodium (III) and proflavine.

To said non-polymeric photosensitive compound can advantageously be associated at least one electron donor selected in particular from vitamin C, ferrocene, 8-hydroxyquinoline-5-sulphonic acid hydrate and a quinone, said electron donor being capable, once oxidised by said photosensitive compound, of being reduced at the surface of the biocathode.

The photosensitive agent may also be at least one photosynthetic protein selected in particular from ferrodoxin and ferrodoxin-NADP reductase.

In a particular embodiment:
(a) aldose reductase; or
(b) aldose reductase and its cofactor NADPH; or
(c) aldose reductase and its cofactor NADPH and at least one regeneration agent for said cofactor, and optionally at least one electron donor in case the regeneration agent is a photosensitive regeneration agent and is a non-polymeric photosensitive compound,
   may be encapsulated in a protective shell capable of letting the reagents and reaction products pass through, but not letting (a), (b) or (c) pass through.

In case the regeneration agent(s) is (are) at least one redox polymer, the aldose reductase and its cofactor may be enclosed in said redox polymer(s), which act(s) as a protective shell, and may be arranged as a layer deposited on the conductive particles.

The protective shell can be made of chitosan, Nafion, polypyrrole, polyacrylic acid.

The invention also relates to a method of manufacturing a biocathode as defined above, characterised by the fact that:
(A) on a collector conductor support, conductive particles are fixed by spraying or printing an ink or paste based on these particles dispersed in water and a surfactant or a polymer or a gel, and then drying said ink or paste; and then
(B) said conductive particles are deposited on:
   (a) an aldose reductase, or
   (b) an aldose reductase and its cofactor NADPH, or
   (c) an aldose reductase, its cofactor NADPH and a regeneration agent for the cofactor,
   at least one of (a), (b) and (c) being capable of being deposited in an encapsulated state in a shell capable of letting the reagents and reaction products pass through but not letting (a), (b) or (c) pass through respectively, or
   it being possible that an encapsulation step be then performed to encapsulate (a), (b) or (c).

In step (B), when the regeneration agent for the cofactor is a redox polymer, the latter can be deposited on the conductive particles by electropolymerisation or electrodeposition or another electrochemical method such as cyclic voltammetry or chronoamperometry or chronopotentiometry, when to aldose reductase is associated its cofactor NADPH, possibly with a protein or proteins, it being possible that the redox polymer be also deposited by chemical polymerisation processes in the presence of an oxidising element, such as iron chloride.

The present invention also relates to a fuel biocell comprising an anode or bioanode and a biocathode as defined above, or manufactured by the process as defined above.

The fuel can be selected from hydrogen and a biomass compound such as glucose, ethanol, glycerol, cholesterol, aldehyde.

The anode may be a bioanode, using, as a catalyst for the oxidation reaction, at least one of enzymes, abiotic compounds, microbes and molecular catalysts.

The fuel biocell according to the invention may be implantable in a human or animal body, for example subcutaneously or in tissue to power an electrical implantable medical device, and optionally externally rechargeable with glucose, monosaccharide, ketone or aldehyde via an external injection of a glucose, monosaccharide, ketone or aldehyde solution.

In particular, it may be implantable in the intestine to be used to consume or quantify glucose, ethanol, glycerol, cholesterol, a monosaccharide, a ketone, an aldehyde, or to generate electrical power.

It can comprise a cathode using glucose as oxidant and an anode using glucose as reductant, without the use of dioxygen.

The biopile may include an anode based on a conductive material such as platinum, gold, graphite, for producing dioxygen in vivo, by connecting the biocathode and the anode to an electrical generator, such as a battery or a lithium cell.

It may be able for operation in anaerobic conditions, mines, sea, space.

The present invention finally relates to a biosensor for glucose, monosaccharide, ketone or aldehyde comprising an anode consisting of a platinum wire and a biocathode as defined above or manufactured by a process as defined above, for in vivo (implantable biosensor) and in vitro applications, means for measuring the value of the reduction current of the glucose, monosaccharide, ketone or aldehyde being provided for estimating the level of glucose, monosaccharide, ketone or aldehyde.

The glucose reduction current is measured at a low potential away from the interfering oxidation potential (potential below OV relative to SCE) and, in this case, the biocathode response is insensitive to the presence of interfering compounds, such as ascorbic acid or dopamine.

To better illustrate the object of the present invention, several embodiments are described below, by way of indication and not as a limitation, with reference to the attached drawing.

On this drawing:

FIG. 1 is a schematic representation of the operation of an enzymatic biofuel cell;

FIG. 2 is a schematic representation of the operation of a biocell with a biocathode according to the invention;

FIG. 3 is a schematic representation of the oxidation of interfering molecules for existing glucose biosensors;

FIG. 4 is a schematic representation of the reduction of a glucose biosensor with a biocathode according to the invention without reduction of interfering molecules;

FIG. 5 is a schematic representation of a biocathode according to a first embodiment with electro-regeneration of the enzymatic cofactor;

FIG. 6 is a schematic representation of a biocathode according to another embodiment with electro-regeneration of the enzymatic cofactor;

FIG. 7 is a schematic representation of a biocathode according to another embodiment with electro-regeneration of the enzymatic cofactor;

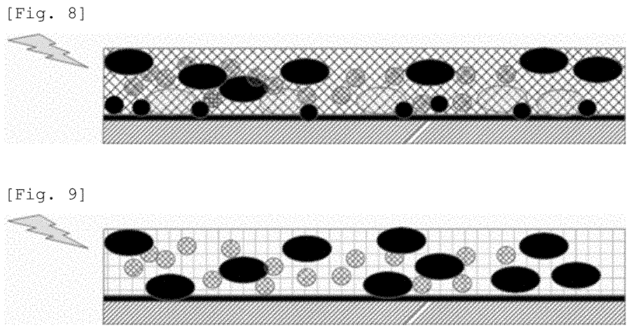

FIG. 8 is a schematic representation of a biocathode in another embodiment with photo-regeneration of the enzyme cofactor;

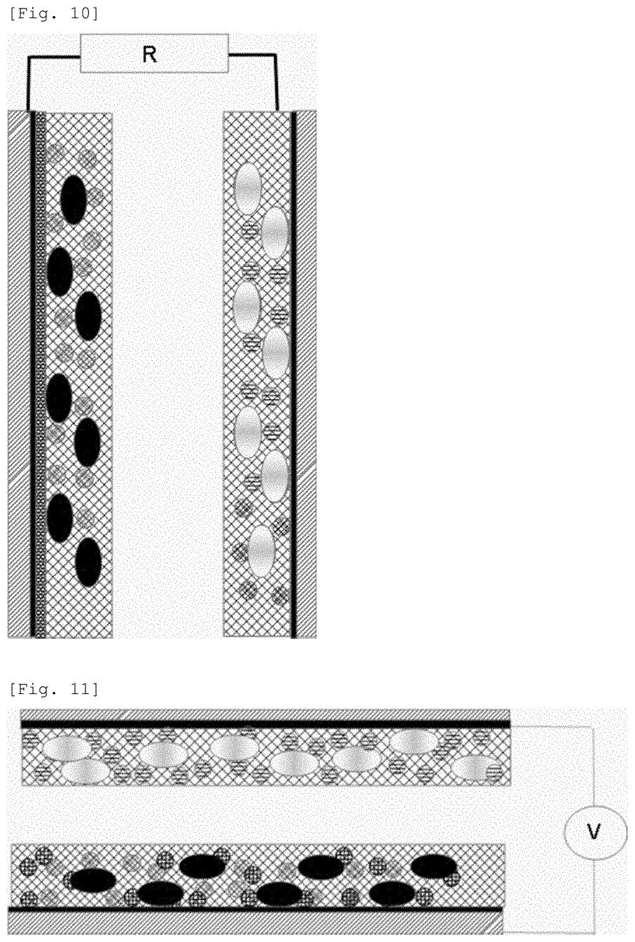

FIG. 9 is a schematic representation of a biocathode in another embodiment with photo-regeneration of the enzyme cofactor;

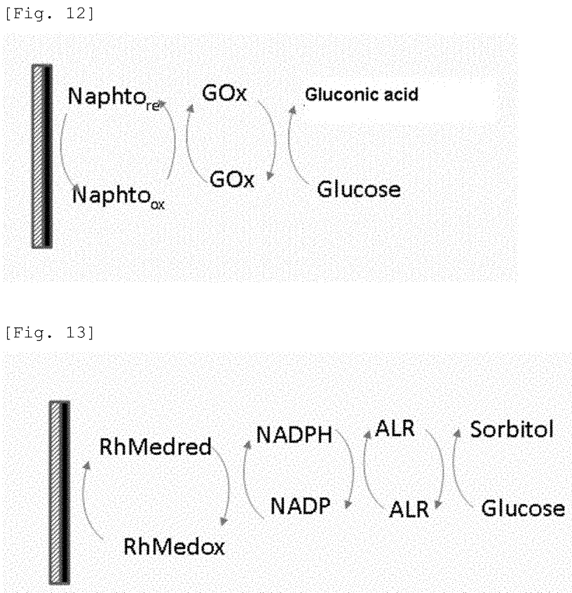

FIG. 10 is a schematic representation of a glucose biocell with a biocathode in a first variant;

FIG. 11 is a schematic representation of a glucose biocell with a biocathode in a second variant;

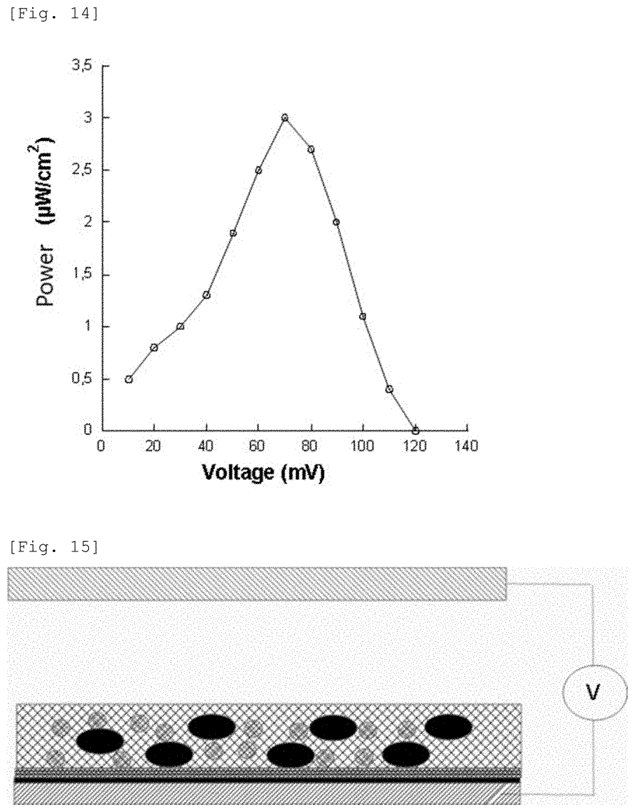

FIG. 12 is a schematic representation of the reactions taking place at the anode during the operation of the biocell shown in FIG. 11;

FIG. 13 is a schematic representation of the reactions taking place at the biocathode during the operation of the biocell shown in FIG. 11;

FIG. 14 is a power versus voltage curve for the biocell shown in FIG. 11 at a glucose concentration of 20 mM in phosphate buffer at pH 7;

FIG. 15 is a schematic representation of a glucose biosensor with a biocathode according to the present invention; and

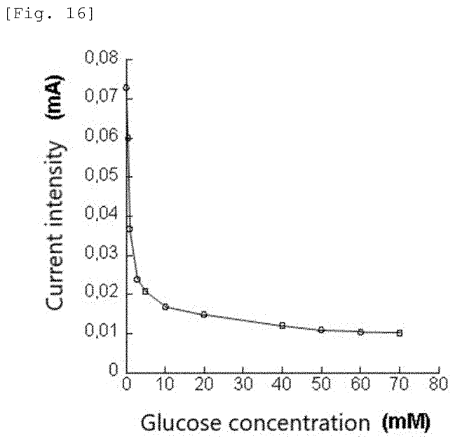

FIG. 16 is a curve of current intensity measured with the biosensor of FIG. 15 as a function of glucose concentration.

In the figures, the following legend is used:
[symbol] Cofactor NADPH
[symbol] Enzyme: aldose reductase
[symbol] Polymer for encaspulation of enzyme and mediator (nafion, chitosan)
[symbol] Layer of conductive particles (carbons, metals, . . . )
[symbol] Conductor support
[symbol] Redox mediator for the regeneration of NADPH
   ((ex: pentamethylcyclopentadienyl-2,2V-bipyridine aqua)rhodium (III)
[symbol] Redox polymer
[symbol] Light
[symbol] Photosensitive molecule (ex. chlorophyll)
● Electron acceptor (ex. Vitamin C)
[symbol] Photosensitive polymer
[symbol] Enzyme 2 for oxidation of glucose (ex: Glucose oxidase)
[symbol] Redox mediator for enzyme 2 (ex: Naphthoquinone, ferrocene, osmium complex)
[symbol] Counter electrode (gold, platinum)

The following examples illustrate the present invention without limiting its scope.

### Example 1: Production of a Biocathode with Electroregeneration of the Cofactor at the Surface of the Electrode

A carbon particle ink is prepared in an aqueous solution containing 0.5% by weight of Tween80 and 5 to 10 mg/mL of carbon particles.

This ink is deposited on a carbon sheet.

After drying under vacuum for two hours, a layer of poly(methylene blue) is deposited on the carbon layer by electropolymerisation.

After rinsing with water, a 1 wt. % Nafion solution containing aldose reductase (100 μM) and its cofactor NADPH (1 mM) is applied and left to dry at room temperature for one hour.

The obtained biocathode is shown schematically in FIG. 5.

### Example 2: Production of a Biocathode with Electroregeneration of the Cofactor Using a Redox Mediator

A carbon particle ink (5-10 mg/mL) is prepared in an aqueous solution containing 0.5 wt % Tween80.

This ink is deposited on a carbon sheet.

A 2 wt. % solution of chitosan containing aldose reductase (100 μM), its cofactor NADPH (1 mM) and a redox mediator pentamethylcyclopentadienyl-2,2V-bipyridine aqua) rhodium (III) (25 μM) is deposited on the carbon sheet, and then left to dry for 6 hours.

The obtained biocathode is shown schematically in FIG. 6.

### Example 3: Production of a Biocathode with Electroregeneration of the Cofactor Using a Redox Polymer

A carbon particle ink (5-10 mg/mL) is prepared in an aqueous solution containing 0.5 wt % Tween80.

This ink is deposited on a carbon sheet.

After drying under vacuum for two hours, a layer of methylene green is electrodeposited on the carbon layer by cyclic voltametry.

A 2 wt. % solution of chitosan containing aldose reductase (100 μM), its cofactor NADPH (1 mM) is deposited on the methylene green layer, and then allowed to dry for 6 hours.

The obtained biocathode is shown schematically in FIG. 7.

### Example 4: Production of a Biocathode with Photoregeneration of the Cofactor Using a Photosensitive Molecule

A carbon particle ink is prepared in an aqueous solution containing 0.5% by weight of Tween80.

This ink is deposited on a carbon sheet.

After drying under vacuum for two hours, a 1% by volume Nafion solution containing aldose reductase (100 μM), its cofactor NADPH (1 mM), ferrodoxin-NADP protein (100 μM), chlorophyll (100 μM) and vitamin C is applied.

The obtained biocathode is shown schematically in FIG. 8.

### Example 5: Production of a Biocathode with Photoregeneration of the Cofactor Using a Photosensitive Polymer

A carbon particle ink is prepared in an aqueous solution containing 0.5% by weight of Tween80.

This ink is deposited on a carbon sheet.

After drying under vacuum for two hours, a 1% by volume solution of Nafion containing aldose reductase (100 μM) and its cofactor NADPH (1 mM) is applied to this carbon sheet and then left to dry for one hour.

The obtained biocathode is shown schematically in FIG. 9.

### Example 6: Production of 100% Glucose Biocell

### Production of a Bioanode

A carbon particle ink is prepared in an aqueous solution containing 0.5% by volume of Tween80.

This ink is deposited on a carbon sheet.

After drying under vacuum for two hours, a 2 wt. % solution of chitosan containing glucose oxidase (100 μM), its mediator naphthoquinone, is applied and everything is left to dry on air at room temperature for six hours.

### Production of the Biocell

A 100% glucose biopile is then produced using the bioanode made above and a biocathode according to Example 3. This biocell oxidises glucose to glucolactone at the bioanode using the enzyme glucose oxidase and its mediator naphthoquinone and reduces glucose to sorbitol at the biocathode.

The obtained biocell is shown schematically in FIG. 10. In the scheme, the current produced by the biocell flows through a resistor R.

### Example 7: Production of 100% Glucose Biocell

### Production of the Biocell

A 100% glucose biocell is made using the bioanode made in Example 6 and a biocathode according to Example 2. This biopile oxidises glucose to gluconic acid at the bioanode using the enzyme glucose oxidase and its mediator naphthoquinone and reduces glucose to sorbitol at the biocathode.

The resulting biopile is shown schematically in FIG. 11. In the scheme, the current produced by the biocell flows through a voltmeter V.

FIGS. 12 and 13 show schematically the reactions occurring at the anode and biocathode, respectively.

At the anode, glucose is oxidised to gluconic acid by the action of glucose oxidase (GOx).

The mediator of glucose oxidase, in the represented example naphthoquinone (Naphto), is oxidised at the anode surface from its reduced form Naphtoᵣₑ to its oxidised form Naphtoₒₓ.

By doing this, electron transfer from the glucose to the bioanode can take place.

At the biocathode, glucose is reduced to sorbitol by the action of aldose reductase and its cofactor NADPH.

The NADPH cofactor is regenerated from its NADP form to its NADPH form using the redox mediator pentamethylcyclopentadienyl-2,2V-bipyridine aqua) rhodium (III), denoted RhMed, in FIG. 13, with RhMedred representing the reductant and RhMedox the oxidant of the redox couple.

The redox mediator is reduced at the cathode surface from its RhMedox form to its RhMedred form.

In this way, electrons are transferred from the biocathode to the glucose so that it can be reduced to sorbitol.

The curve of FIG. 14 shows the characteristics of the resulting biocell. The voltage of the open circuit biocell is 120 mV and it is capable of producing a power density of 3 μW/cm² at a glucose concentration of 20 mM.

### Example 8: Production of a Glucose Biosensor

A glucose biosensor is made using a biocathode according to Example 3 and a conventional counter electrode such as a gold or platinum counter electrode.

The resulting biosensor is shown schematically in FIG. 15. In the scheme, the current produced by the biocell flows through a voltmeter V.

From this biosensor, a calibration curve at zero voltage of the intensity measured using the biosensor versus glucose concentration can be obtained. This curve is shown in FIG. 16.

1. A biomass-based enzymatic biocathode based on one of monosaccharide, ketone and aldehyde, comprising:
a collector conductor support;
conductive particles disposed on and bound to the collector conductor support;
an aldose reductase disposed on the conductive particles, being bound thereto by adsorption and accessible at the surface for the monosaccharide, ketone or aldehyde reagent that is to be reduced when the biocathode is operational.

2. The enzymatic biocathode according to claim 1, wherein the collector conductor support is selected from:
continuous sheets of one of carbon, graphene and graphite;
continuous sheets of a metal;
continuous indium tin oxide (ITO) sheets; and
carbon fibre non-woven fabrics.

3. The enzymatic biocathode according to claim 1, wherein the conductive particles are selected from particles of carbon, graphene, graphite, carbon black or mesoporous carbon nanotubes, and particles of multiwalled carbon nanotubes (MWCNT).

4. The enzymatic biocathode according to claim 1, to the aldose reductase is associated its nicotinamide adenine dinucleotide phosphate (NADPH) cofactor.

5. The enzymatic biocathode according to claim 24, wherein the regeneration agent is an agent for the electroregeneration of the NADPH cofactor at the surface of the biocathode, the electroregeneration agent being at least one redox polymer chosen from benzylpropylviologen, a viologen polysiloxane polymer, polyaniline or polypyrrole.

6. The enzymatic biocathode according to claim 24, wherein the regeneration agent is a photosensitive agent for the regeneration of the NADPH cofactor at the surface of the biocathode, the photosensitive agent being at least one redox photosensitive polymer chosen from methylene green, methylene blue, neutral red, and polyaniline and polypyrrole.

7. The enzymatic biocathode according to claim 24, wherein the regeneration agent is a photosensitive agent for the regeneration of the NADPH cofactor at the surface of the biocathode, the photosensitive agent being at least one non-polymeric photosensitive compound selected from among chlorophyll, acridine, (pentamethylcyclopentadienyl-2,2V-bipyridine aqua) rhodium (III) and proflavine.

8. The enzymatic biocathode according to claim 7, wherein to the non-polymeric photosensitive compound is associated at least one electron donor selected from vitamin C, ferrocene, 8-hydroxyquinoline-5-sulphonic acid hydrate and a quinone, the electron donor being capable, once oxidized by said photosensitive compound, of being reduced at the surface of the biocathode.

9. The enzymatic biocathode according to claim 24, wherein the regeneration agent is a photosensitive agent for the regeneration of the NADPH cofactor at the surface of the biocathode, the photosensitive agent being at least one photosynthesis protein selected from ferrodoxin and ferrodoxin-NADP reductase.

10. The enzymatic biocathode according to claim 1, wherein:
(a) aldose reductase; or
(b) aldose reductase and its cofactor NADPH; or
(c) aldose reductase and its cofactor NADPH and at least one regeneration agent for said cofactor, and optionally at least one electron donor in case the regeneration agent is a photosensitive regeneration agent and is a non-polymeric photosensitive compound is/are encapsulated in a protective shell capable of letting the reagents and reaction products pass through, but not letting (a), (b) or (c) pass through.

11. The enzymatic biocathode according to claim 10, wherein the regeneration agent(s) is (are) at least one redox polymer, the aldose reductase and its cofactor being enclosed in said redox polymer(s), which act(s) as a protective shell, and can be arranged in the form of a layer deposited on the conductive particles.

12. The enzymatic biocathode according to claim 11, wherein the protective shell is made of chitosan, Nafion, polypyrrole, polyacrylic acid.

13. A method of manufacturing a biocathode wherein:
(A) on a collector conductor support, conductive particles are fixed by spraying or printing an ink or paste based on these particles dispersed in water and a surfactant or a polymer or a gel, and then drying said ink or paste; and then
(B) said conductive particles are deposited on:
(a) an aldose reductase, or
(b) an aldose reductase and its cofactor NADPH, or
(c) an aldose reductase, its cofactor NADPH and a regeneration agent for the cofactor,
at least one of (a), (b) and (c) being capable of being deposited in an encapsulated state in a shell capable of letting the reagents and reaction products pass through but not letting (a), (b) or (c) pass through respectively, or
it being possible that an encapsulation step be then performed to encapsulate (a), (b) or (c).

14. The method according to claim 13, wherein in step (B), when the regeneration agent for the cofactor is a redox polymer, the latter is deposited on the conductive particles by electropolymerization or electrodeposition or another electrochemical method such as cyclic voltammetry or chronoamperometry or chronopotentiometry, when to aldose reductase is associated its cofactor NADPH, possibly with a protein or proteins, it being possible that the redox polymer be also deposited by chemical polymerisation processes in the presence of an oxidising element, such as iron chloride.

15. A fuel biocell comprising an anode or bioanode and a biocathode as defined in claim 1.

16. The fuel biocell according to claim 15, wherein the fuel is selected from hydrogen and a biomass compound selected from glucose, ethanol, glycerol, cholesterol, an aldehyde.

17. The fuel biocell according to claim 15, wherein the anode is a bioanode, using, as a catalyst for the oxidation reaction, at least one of enzymes, abiotic compounds, microbes and molecular catalysts.

18. The fuel biocell according to claim 17, wherein it is implantable in a human or animal body, for example subcutaneously or in tissue to power an electrically implantable medical device, and optionally externally rechargeable with glucose, monosaccharide, ketone or aldehyde by means of an external injection of a glucose, monosaccharide, ketone or aldehyde solution.

19. The fuel biocell of claim 18, wherein it is implantable in the intestine so as to be used to consume or quantify glucose, ethanol, glycerol, cholesterol, a monosaccharide, a ketone, an aldehyde, or to generate electrical power.

20. The fuel biocell according to claim 15, wherein it comprises a cathode using glucose as oxidant and an anode using glucose as reductant, without the use of dioxygen.

21. The fuel biocell according to claim 18, wherein it comprises an anode based on a conductive material such as platinum, gold, graphite, for producing dioxygen in vivo, by connecting the biocathode and the anode to an electrical generator.

22. The fuel biocell according to claim 15, wherein it is suitable for operation in anaerobic conditions, mines, sea, space.

23. A biosensor for glucose, monosaccharide, ketone or aldehyde comprising an anode consisting of a platinum wire and a biocathode as defined in claim 1, for in vivo and in vitro applications, means for measuring the value of the reduction current of the glucose, monosaccharide, ketone or aldehyde being provided for estimating the level of glucose, monosaccharide, ketone or aldehyde.

24. The enzymatic biocathode according to claim 4, wherein it comprises at least one agent for the regeneration of the NADPH cofactor by catalyzing its reduction at the surface of the biocathode, the regeneration agent allowing an electro- or a photo-regeneration, being in this case photosensitive.
